(19) United States
(12) Patent Application Publication
(10) Pub. No.: US 2018/0271490 A1
(43) Pub. Date: Sep. 27, 2018

(54) ULTRASOUND TRANSDUCER UNIT, ULTRASOUND PROBE, AND METHOD OF PRODUCING ULTRASOUND TRANSDUCER UNIT

(71) Applicant: OLYMPUS CORPORATION, Tokyo (JP)

(72) Inventor: Toshihiro KITAHARA, Tokyo (JP)

(73) Assignee: OLYMPUS CORPORATION, Tokyo (JP)

(21) Appl. No.: 15/994,147

(22) Filed: May 31, 2018

Related U.S. Application Data
(63) Continuation of application No. PCT/JP2016/084242, filed on Nov. 18, 2016.

(30) Foreign Application Priority Data
Dec. 2, 2015 (JP) 2015-236129

(51) Int. Cl. A61B 8/00; A61B 8/12; A61B 8/14

(52) U.S. Cl. CPC A61B 8/4461; A61B 8/14; A61B 8/12

(57) ABSTRACT

An ultrasound transducer unit includes: a transducer section including an ultrasound transducer including a plurality of piezoelectric elements arranged in an array; and a housing configured to house the transducer section such that the transducer section is rotatable along an arrangement direction of the plurality of piezoelectric elements.

### CROSS-REFERENCE TO RELATED APPLICATION

This application is a continuation of PCT international application Ser. No. PCT/JP2016/084242 filed on Nov. 18, 2016 which designates the United States, incorporated herein by reference, and which claims the benefit of priority from Japanese Patent Applications No. 2015-236129, filed on Dec. 2, 2015, incorporated herein by reference.

### BACKGROUND

The present disclosure relates to an ultrasound transducer unit, an ultrasound probe, and a method of producing an ultrasound transducer unit.

An ultrasound transducer unit is known to have an ultrasound transducer disposed at a distal end of an insertion section inserted into a subject's body (e.g., see JP 2002-199494 A). In such an ultrasound transducer unit, a transducer section including an ultrasound transducer, a substrate, a signal cable, and the like are housed in a housing.

### SUMMARY

An ultrasound transducer unit according to one aspect of the present disclosure includes: a transducer section including an ultrasound transducer including a plurality of piezoelectric elements arranged in an array; and a housing configured to house the transducer section such that the transducer section is rotatable along an arrangement direction of the plurality of piezoelectric elements.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram of an endoscope system according to a first embodiment;

FIG. 2 is a perspective view of an ultrasound transducer unit;

FIG. 3 is a diagram of a transducer section of FIG. 2 from which an acoustic lens is removed;

FIG. 4 is a side view of the transducer section of FIG. 3 viewed from above;

FIG. 5 is an enlarged perspective view of a housing of FIG. 2;

FIG. 6 is a diagram illustrating positioning of the transducer section to the housing;

FIG. 7 is a perspective view of a transducer section of an ultrasound transducer unit according to modification 1-1;

FIG. 8 is a perspective view of an ultrasound transducer unit according to a second embodiment, in which a transducer section is disassembled from a housing;

FIG. 9 is a perspective view of an ultrasound transducer unit according to modification 2-1, in which a transducer section is disassembled from a housing;

FIG. 10 is a cross-sectional view of an ultrasound transducer unit according to a third embodiment;

FIG. 11 is a perspective view of a transducer section of FIG. 10, which is disassembled from a housing;

FIG. 12 is an enlarged perspective view of the housing of FIG. 10;

FIG. 13 is a cross-sectional view of an ultrasound transducer unit according to modification 3-1;

FIG. 14 is a perspective view of a transducer section of FIG. 13, which is disassembled from a housing; and

FIG. 15 is an enlarged perspective view of the housing of FIG. 13.

### DETAILED DESCRIPTION

Hereinafter, embodiments of an ultrasound transducer unit, an ultrasound probe, and a method of producing an ultrasound transducer unit according to the present disclosure will be described with reference to the drawings. Note that the present disclosure is not limited to these embodiments. The present disclosure is generally applicable to ultrasound transducer units including a transducer section with an ultrasound transducer and a housing for storing the transducer section.

Furthermore, in the drawings, the same or corresponding elements are appropriately denoted by the same reference signs. In addition, each drawing is schematically illustrated, and dimensional relationships, ratios, and the like between the elements may be different from actual dimensional relationships, ratios, and the like. Some drawings may include portions having different dimensional relationships and ratios between the drawings.

### First Embodiment

Outline of Configuration of Endoscope System

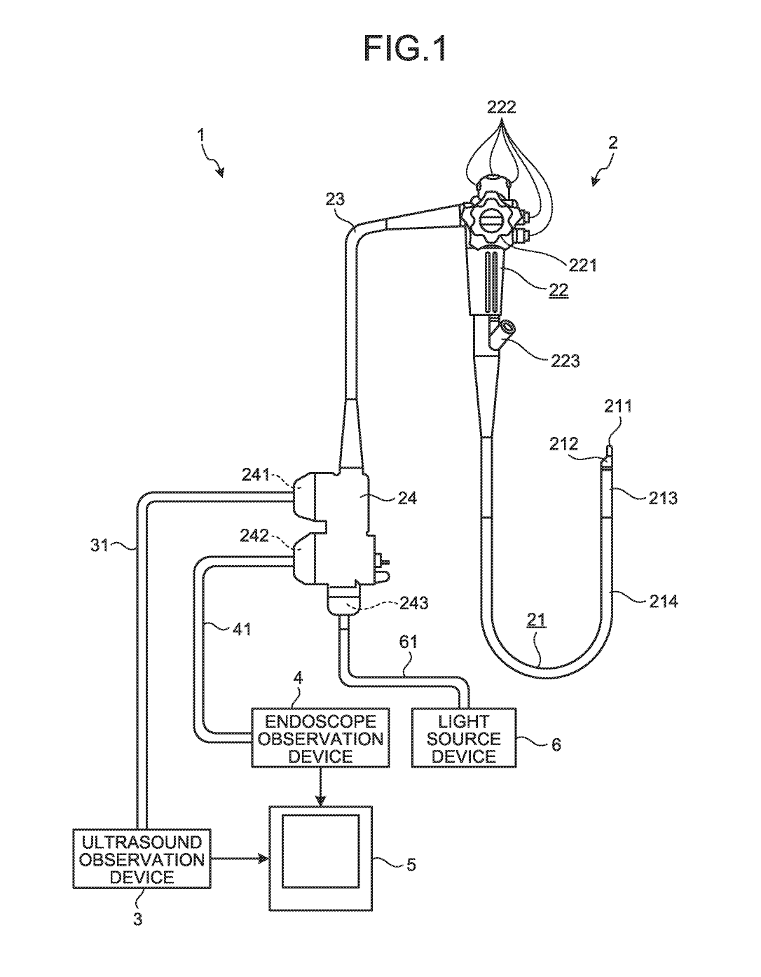

FIG. 1 is a schematic diagram of an endoscope system according to a first embodiment. An endoscope system 1 is a system performing ultrasound diagnosis in a subject, such as a human, using an ultrasound endoscope. As illustrated in FIG. 1, the endoscope system 1 includes an endoscope 2, an ultrasound observation device 3, an endoscope observation device 4, a display device 5, and a light source device 6. The endoscope 2 is an ultrasound endoscope configured to be partially inserted into the subject, and having a function of transmitting an ultrasound pulse toward a body wall in the subject, receiving an ultrasound echo reflected from the subject, and outputting an echo signal, and a function of imaging inside the subject and outputting an image signal. Note that a detailed configuration of the endoscope 2 will be described later.

The ultrasound observation device 3 is electrically connected to the endoscope 2 through an ultrasound cable 31, and outputs a pulse signal to the endoscope 2 and receives input of an echo signal from the endoscope 2 through the ultrasound cable 31. Then, the ultrasound observation device 3 performs predetermined processing on the echo signal to generate an ultrasound image.

The endoscope observation device 4 is electrically connected to the endoscope 2 through a video cable 41, and receives input of an image signal from the endoscope 2 through the video cable 41. Then, the endoscope observation device 4 performs predetermined processing on the image signal to generate an endoscopic image.

The display device 5 includes liquid crystal or organic electro luminescence (EL), and displays an ultrasound image generated by the ultrasound observation device 3, an endoscopic image generated by the endoscope observation device 4, or the like.

The light source device 6 is connected to the endoscope 2 through an optical fiber cable 61, and supplies illumination light illuminating inside the subject to the endoscope 2 through the optical fiber cable 61.

Configuration of Endoscope

As illustrated in FIG. 1, the endoscope 2 includes an insertion section 21, an operating unit 22, a universal cable 23, and a connector 24. Note that “distal end” described below represents an end portion positioned on a distal end side of the insertion section 21. In addition, “proximal end” described below represents an end portion positioned on a side away from the distal end of the insertion section 21 (on the side of the operating unit 22).

The insertion section 21 is a portion inserted into the subject. As illustrated in FIG. 1, the insertion section 21 includes an ultrasound transducer unit 211 provided on the distal end side, a rigid member 212 connected to the proximal end side of the ultrasound transducer unit 211, a bending section 213 bendably connected on the proximal end side of the rigid member 212, and a flexible tube portion 214 flexibly connected to the proximal end side of the bending section 213.

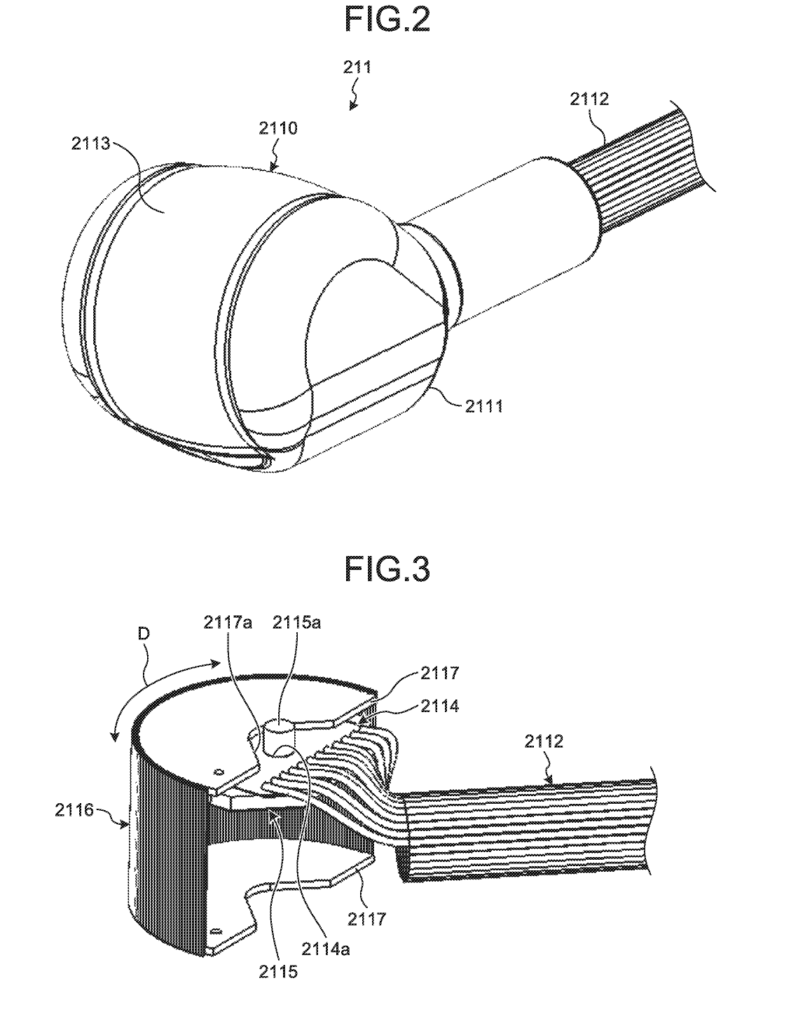

Here, in the insertion section 21, a light guide (not illustrated), an image guide (not illustrated), a plurality of signal cables, and a tube (not illustrated) are routed. The light guide transmits illumination light supplied from the light source device 6, the image guide conducts an optical image captured inside the subject, which is described later, the plurality of signal cables transmits various signals (e.g., a signal cable 2112 (see FIG. 2) electrically connected to the ultrasound cable 31 through the universal cable 23, which is described later), and the tube receives insertion of various treatment instruments (not illustrated). Note that detailed configuration of the distal end side of the ultrasound transducer unit 211 will be described later.

The bending section 213 is a portion having a cylindrical shape and bent in accordance with operation of a bending knob 221 by a physician or the like.

The rigid member 212 functions as a distal end member according to the present disclosure, is a rigid member made of a resin material, and has a substantially columnar shape.

The operating unit 22 is a portion connected to the proximal end of the insertion section 21 to receive various operations from the physician or the like. As illustrated in FIG. 1, the operating unit 22 includes the bending knob 221 for bendably operating the bending section 213, and a plurality of operation members 222 for performing various operations. Further, in the operating unit 22, a treatment instrument insertion opening 223 is formed. The treatment instrument insertion opening 223 communicates with the tube (not illustrated) disposed in the insertion section 21 to insert the various treatment instruments through the tube. Still further, in the operating unit 22, an imaging sensor (not illustrated) and an optical system (not illustrated) are disposed. The imaging sensor outputs an image signal in accordance with an optical image captured inside the subject, and the optical system forms, on the imaging sensor, an optical image conducted by the image guide.

The universal cable 23 is a cable connected to the operating unit 22 at one end. In the universal cable 23, a plurality of signal cables transmitting various signals, an optical fiber for transmitting illumination light supplied from the light source device 6, and the like are disposed.

The connector 24 is provided at the other end of the universal cable 23. The connector 24 includes first to third connector portions 241 to 243 to which the ultrasound cable 31, the video cable 41, and the optical fiber cable 61 are connected respectively.

Configuration of Ultrasound Transducer Unit

FIG. 2 is a perspective view of an ultrasound transducer unit. As illustrated in FIG. 2, the ultrasound transducer unit 211 includes a transducer section 2110 being a convex ultrasound probe, and a housing 2111 for storing the transducer section 2110.

Firstly, a configuration of the transducer section 2110 will be described. The transducer section 2110 includes the signal cable 2112 for transmitting a pulse signal from the ultrasound observation device 3 and transmitting an echo signal from the transducer section 2110 to the ultrasound observation device 3, and an acoustic lens 2113 provided on an outer surface of the transducer section 2110.

FIG. 3 is a diagram of the transducer section of FIG. 2 from which the acoustic lens is removed. As illustrated in FIG. 3, the transducer section 2110 includes a substrate 2114, a substrate supporting portion 2115 for supporting the substrate 2114, an ultrasound transducer 2116 having a plurality of piezoelectric elements arranged in an array, and side boards 2117 arranged on both side surfaces of the ultrasound transducer 2116.

The signal cable 2112 has a plurality of cables at a distal end at which each cable is electrically connected to the substrate 2114 by soldering or the like to transmit and receive signals to and from each piezoelectric element of the ultrasound transducer 2116.

The acoustic lens 2113 includes silicone, polymethylpentene, epoxy resin, polyetherimide, or the like, and has one side surface having a convex or concave shape. When outputting an ultrasound wave from the ultrasound transducer 2116 to the outside, the acoustic lens 2113 diffuses the ultrasound wave, and when capturing an ultrasound echo into the ultrasound transducer 2116 from outside, the acoustic lens 2113 concentrates the ultrasound wave. Note that between the acoustic lens 2113 and the ultrasound transducer 2116, one or a plurality of acoustic matching layers may be disposed to match acoustic impedances of the piezoelectric elements and an object to be observed.

The substrate 2114 is formed on one of side surfaces at both ends of the piezoelectric elements to electrically connect the plurality of cables of the signal cable 2112 to the respective piezoelectric elements of the ultrasound transducer 2116. The substrate 2114 includes a hole 2114a configured to receive insertion of a protruding portion 2115a of the substrate supporting portion 2115, which is described later.

The substrate supporting portion 2115 is formed into a flat plate shape and has a main surface for supporting the substrate 2114 thereon. The substrate supporting portion 2115 includes the protruding portion 2115a having a columnar shape projecting in a direction perpendicular to the main surface.

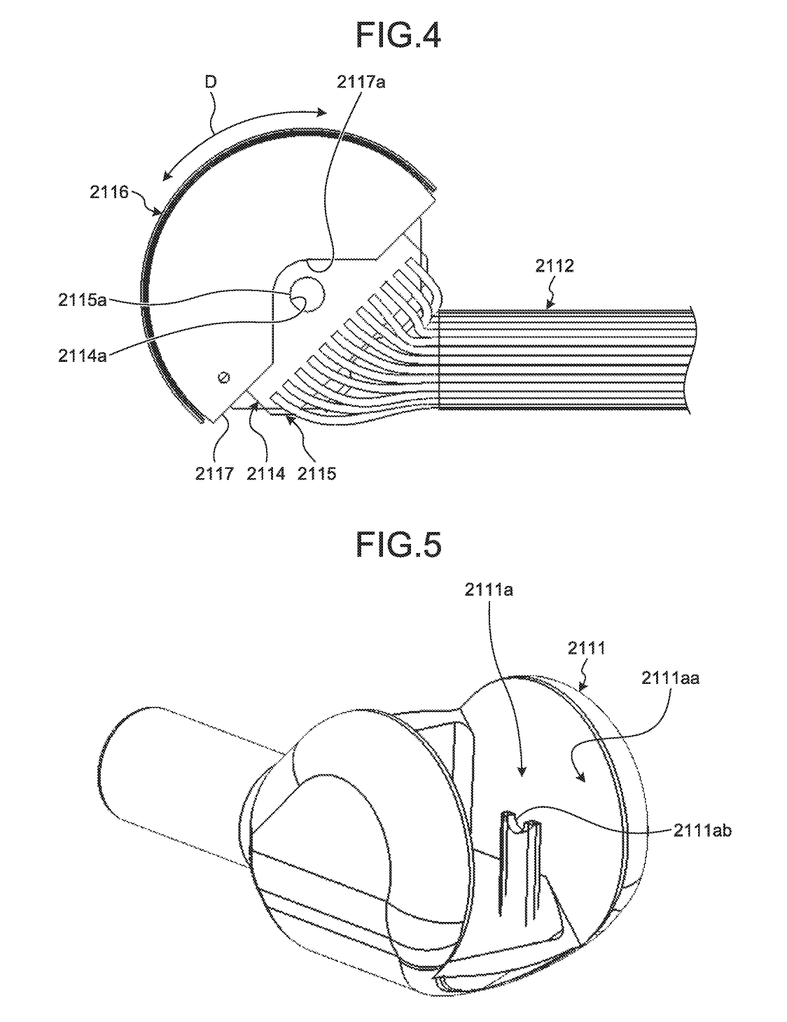

FIG. 4 is a side view of the transducer section of FIG. 3 viewed from above. As illustrated in FIG. 4, the ultrasound transducer 2116 includes the plurality of piezoelectric elements arcuately arranged in an arrangement direction D, and each piezoelectric element transmits an ultrasound pulse to a body wall in the subject, receives an ultrasound echo reflected from the subject, and outputs an echo signal. Here, the protruding portion 2115a is disposed at the center of curvature of the arcuately arranged piezoelectric elements, and protrudes in a direction perpendicular to the arrangement direction D of the piezoelectric elements.

In a side board 2117, a cutout portion 2117a is formed to prevent interference between the side board 2117 and the protruding portion 2115a of the substrate supporting portion 2115.

FIG. 5 is an enlarged perspective view of the housing of FIG. 2. As illustrated in FIG. 5, the housing 2111 includes an opening portion 2111a configured to house the transducer section 2110. The opening portion 2111a has an inner wall surface 2111aa perpendicular to the protruding portion 2115a, facing the protruding portion 2115a, and further extending in the arrangement direction D of the piezoelectric elements. Furthermore, the inner wall surface 2111aa includes a supporting portion 2111ab configured to support the protruding portion 2115a of the substrate supporting portion 2115.

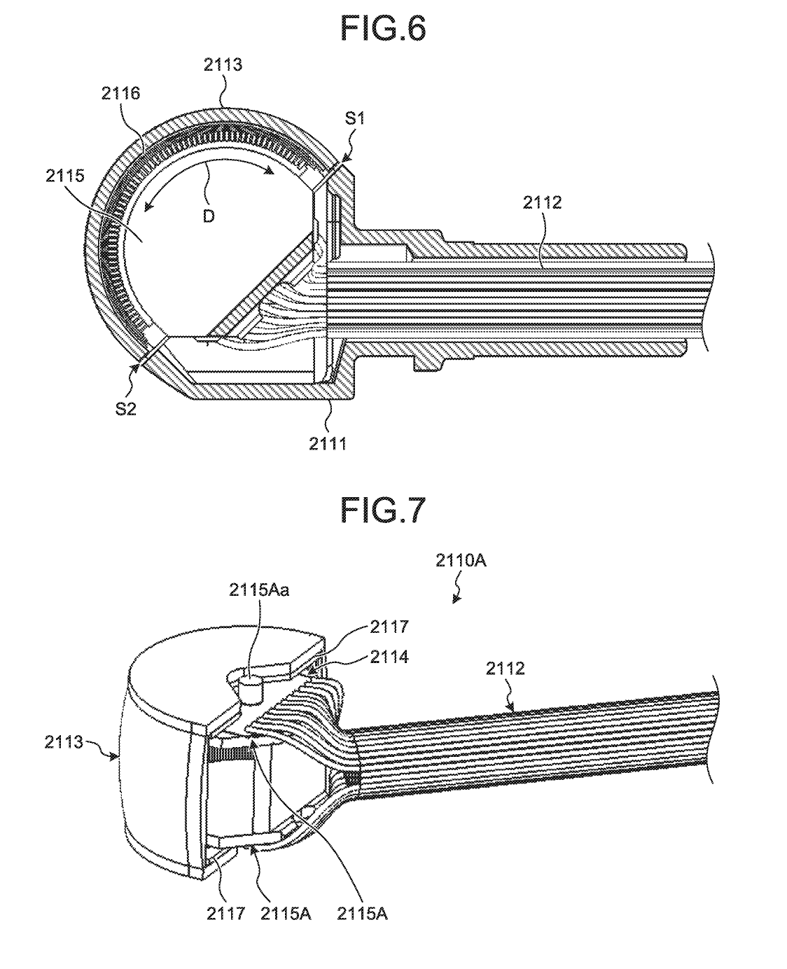

In this ultrasound transducer unit 211, the protruding portion 2115a of the substrate supporting portion 2115 is rotatably supported by the supporting portion 2111ab of the housing 2111, the transducer section 2110 is positioned to the housing 2111, and then the positioned transducer section 2110 is fixed to the housing 2111. FIG. 6 is a diagram illustrating positioning of the transducer section to the housing. FIG. 6 is a cross-sectional view of a state in which the protruding portion 2115a of the substrate supporting portion 2115 is rotatably supported by the supporting portion 2111ab of the housing 2111. As illustrated in FIG. 6, between the transducer section 2110 and the housing 2111, a space S1 and a space S2 are formed. Then, the transducer section 2110 is rotated around the protruding portion 2115a to adjust the space S1 and the space S2 to have the same thickness by using a thickness gauge surely measuring thickness, or the like. Thus, the transducer section 2110 may be accurately positioned to the housing 2111. After the positioning, to prevent a change in thickness of the space S1 and the space S2, the transducer section 2110 is fixed to the housing 2111 with an adhesive or the like.

As described above, the ultrasound transducer unit 211 is an ultrasound transducer unit in which a transducer section is accurately positioned to a housing.

Note that the space S1 and the space S2 may be adjusted to have different thicknesses to position the transducer section 2110 to the housing 2111.

Modification 1-1

FIG. 7 is a perspective view of a transducer section of an ultrasound transducer unit according to modification 1-1. A transducer section 2110A of the ultrasound transducer unit according to modification 1-1 is provided with substrate supporting portions 2115A formed on side surfaces at both ends of piezoelectric elements of the transducer section 2110A. In each substrate supporting portion 2115A, a protruding portion 2115Aa is formed to have a columnar shape projecting outward (a protruding portion 2115Aa positioned on a back side is not illustrated in FIG. 7). Similarly, a housing, not illustrated, has an opening portion, and the opening portion has inner wall surfaces each perpendicular to a corresponding protruding portion 2115Aa and facing the protruding portion 2115Aa. On each inner wall surface, a supporting portion is formed to support a corresponding protruding portion 2115Aa.

### Second Embodiment

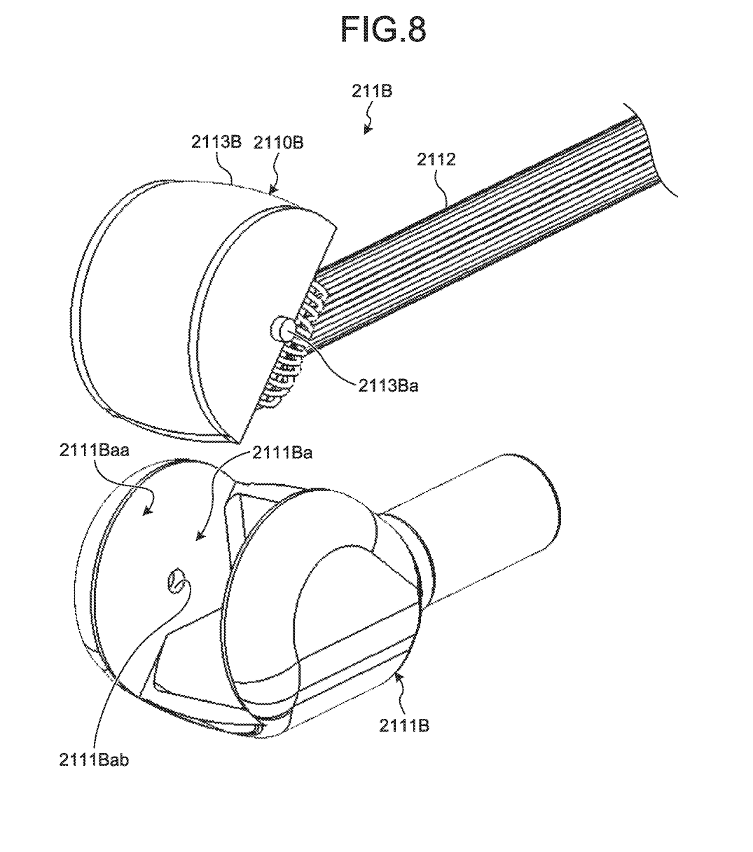

FIG. 8 is a perspective view of an ultrasound transducer unit according to a second embodiment, in which a transducer section is disassembled from a housing. As illustrated in FIG. 8, an ultrasound transducer unit 211B includes a transducer section 2110B and a housing 2111B. The transducer section 2110B has side surfaces at both ends of piezoelectric elements of an acoustic lens 2113B, and a protruding portion 2113Ba of columnar shape is formed on the side surfaces (a protruding portion 2113Ba positioned on a back side is not illustrated in FIG. 8). An opening portion 2111Ba is formed in the housing 2111B to house the transducer section 2110B, the opening portion 2111Ba has inner wall surfaces 2111Baa perpendicular to the protruding portions 2113Ba and facing the protruding portions 2113Ba, and on each inner wall surfaces 2111Baa, a supporting portion 2111Bab (a supporting portion 2111Bab on the front side is not illustrated in FIG. 8) is formed to be fitted to and support a corresponding protruding portion 2113Ba of the acoustic lens 2113B. Note that the acoustic lens 2113B includes an elastic member, such as silicone, and when the transducer section 2110B is fitted into the housing 2111B, each protruding portion 2113Ba is elastically deformed. Then, when each protruding portion 2113Ba is positioned to a corresponding supporting portion 2111Bab, the protruding portion 2113Ba is fitted into the supporting portion 2111Bab. Furthermore, the protruding portion 2113Ba and the supporting portion 2111Bab may be formed on only one of the side surfaces at both ends of the piezoelectric elements.

Modification 2-1

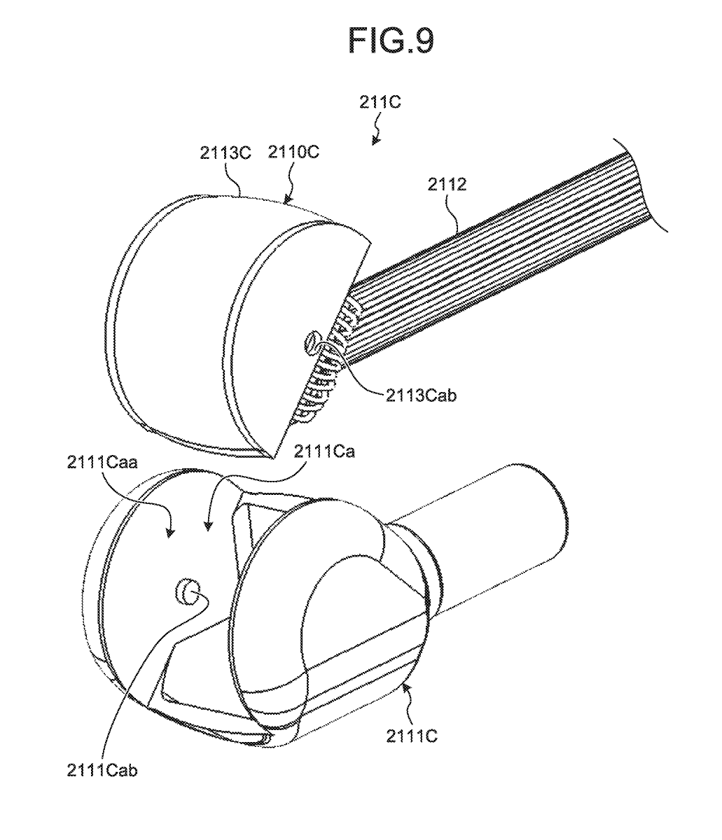

FIG. 9 is a perspective view of an ultrasound transducer unit according to modification 2-1, in which a transducer section is disassembled from a housing. As illustrated in FIG. 9, an ultrasound transducer unit 211C includes a transducer section 2110C and a housing 2111C. An opening portion 2111Ca is formed in the housing 2111C to house the transducer section 2110C. In the opening portion 2111Ca, a protruding portion 2111Cab of columnar shape is formed (a protruding portion 2111Cab on the front side is not illustrated in FIG. 9) on a pair of inner wall surfaces 2111Caa extending in an arrangement direction of piezoelectric elements. An acoustic lens 2113C is provided on an outer surface of an ultrasound transducer of the transducer section 2110C. The acoustic lens 2113C has side surfaces at both ends of the piezoelectric elements, and on the side surfaces a supporting portion 2113Cab is formed (a supporting portion 2113Cab on a back side is not illustrated in FIG. 9) to be fitted to and support a corresponding protruding portion 2111Cab of the housing 2111C. Note that the acoustic lens 2113C includes an elastic member, such as silicone, and when the transducer section 2110C is fitted into the housing 2111C, the acoustic lens 2113C pressed against the protruding portions 2111Cab is elastically deformed. Then, when each protruding portion 2111Cab is positioned to a corresponding supporting portion 2113Cab, the protruding portion 2111Cab is fitted into the supporting portion 2113Cab. Furthermore, the protruding portion 2111Cab and the supporting portion 2113Cab may be formed on only one of the side surfaces at both ends of the piezoelectric elements.

### Third Embodiment

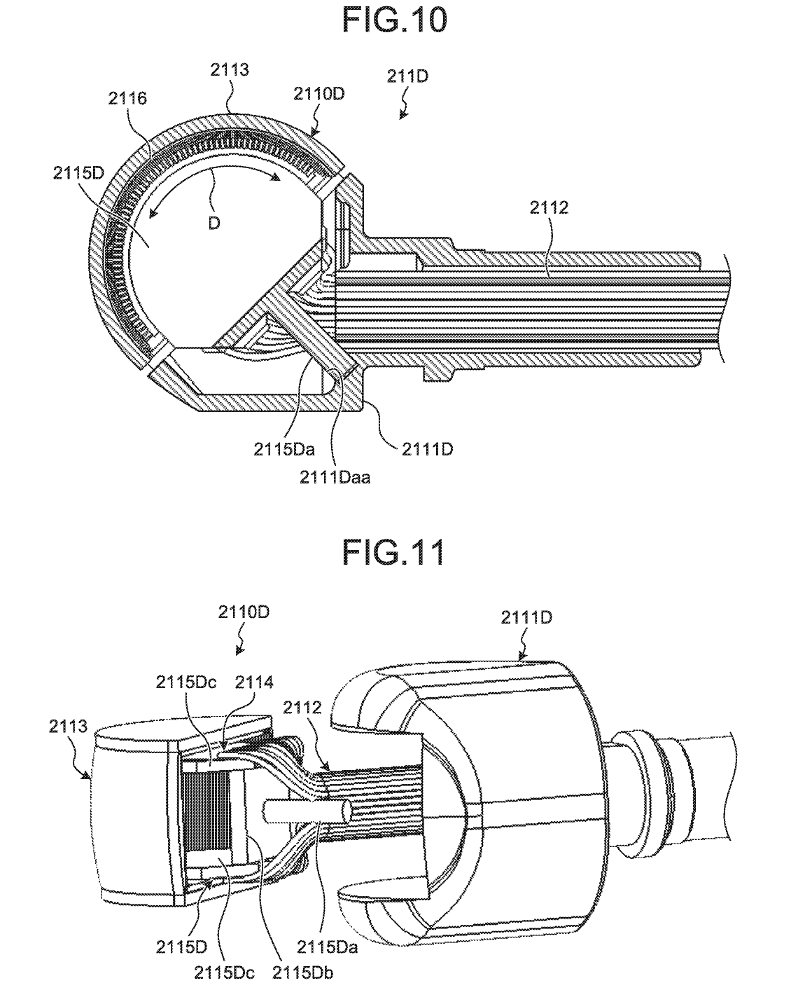

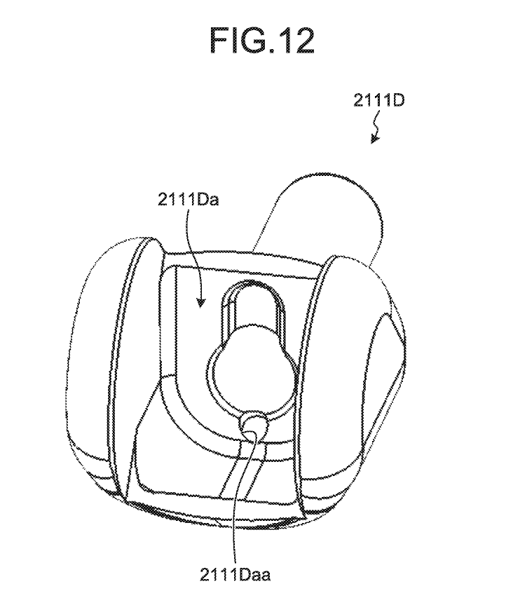

FIG. 10 is a cross-sectional view of an ultrasound transducer unit according to a third embodiment. FIG. 11 is a perspective view of a transducer section of FIG. 10, which is disassembled from a housing. FIG. 12 is an enlarged perspective view of the housing of FIG. 10. As illustrated in FIGS. 10 to 12, an ultrasound transducer unit 211D includes a transducer section 2110D and a housing 2111D. The transducer section 2110D includes a substrate support 2115D in which a cylindrical projecting portion 2115Da is formed to project on a side opposite to an ultrasound transducer 2116 arranged in an array. The substrate support 2115D includes the projecting portion 2115Da, a supporting plate 2115Db for supporting the projecting portion 2115Da, and substrate supporting portions 2115Dc connected to both ends of the supporting plate 2115Db, each formed into a flat plate shape, and each having a main surface for supporting a substrate 2114 thereon. In an opening portion 2111Da formed in the housing 2111D to house the transducer section 2110D, a recess portion 2111Daa into which the projecting portion 2115Da is fitted is formed. In the ultrasound transducer unit 211D, the projecting portion 2115Da and the recess portion 2111Daa are fitted to each other to position the transducer section 2110D to the housing 2111D. Accordingly, the ultrasound transducer unit 211D is an ultrasound transducer unit in which a transducer section is accurately positioned to a housing.

Modification 3-1

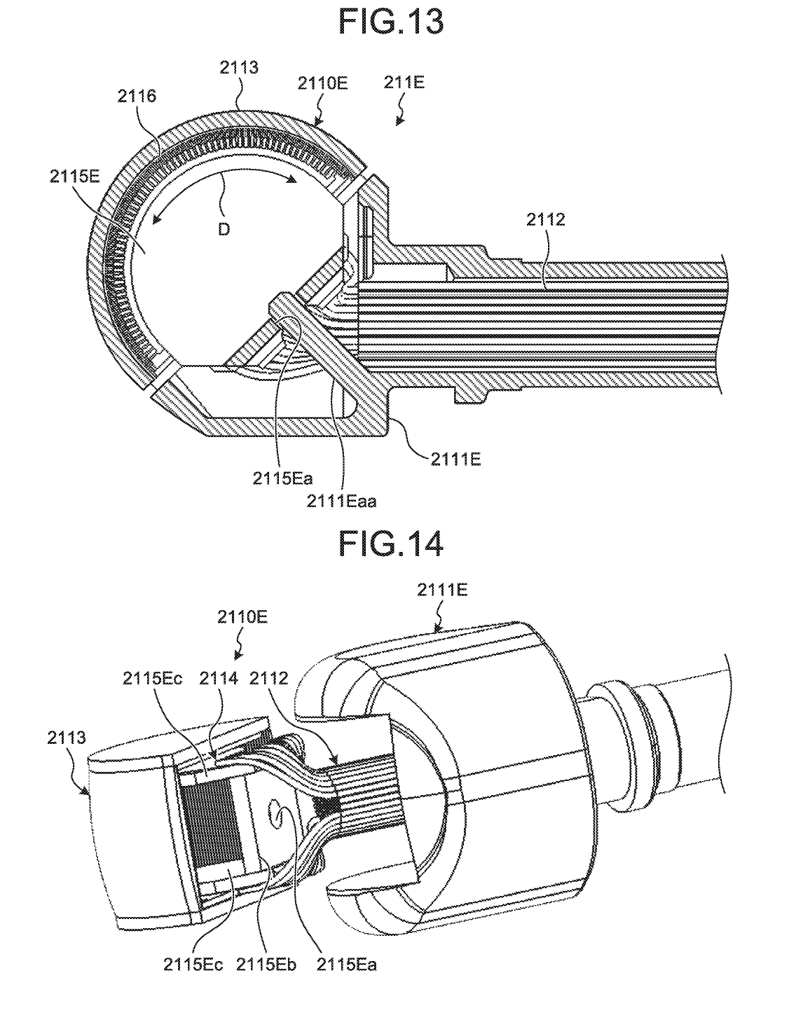

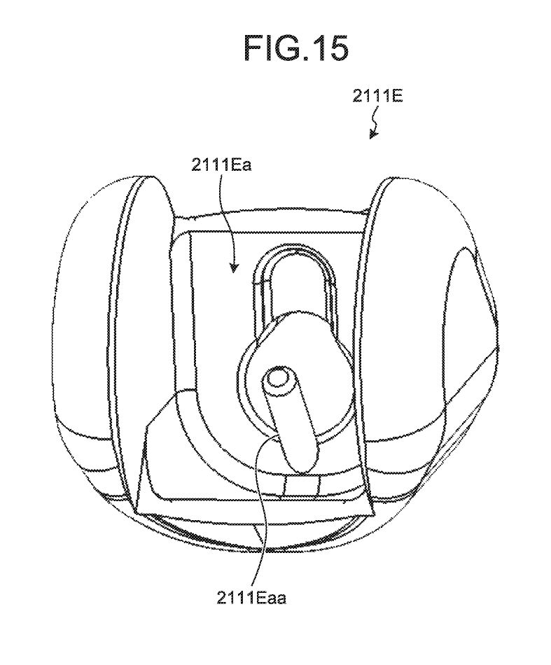

FIG. 13 is a cross-section of an ultrasound transducer unit according to modification 3-1. FIG. 14 is a perspective view of a transducer section of FIG. 13, which is disassembled from a housing. FIG. 15 is an enlarged perspective view of the housing of FIG. 13. As illustrated in FIGS. 13 to 15, an ultrasound transducer unit 211E includes a transducer section 2110E and a housing 2111E. In an opening portion 2111Ea formed in the housing 2111E to house the transducer section 2110E, a cylindrical projecting portion 2111Eaa is formed to project toward an ultrasound transducer 2116 arranged in an array. The transducer section 2110E includes a substrate support 2115E having a recess portion 2115Ea into which the projecting portion 2111Eaa is fitted. The substrate support 2115E includes the recess portion 2115Ea, a supporting plate 2115Eb for supporting the recess portion 2115Ea, and substrate supporting portions 2115Ec connected to both ends of the supporting plate 2115Eb, each formed into a flat plate shape, and each having a main surface for supporting a substrate 2114 thereon. In the ultrasound transducer unit 211E, the projecting portion 2111Eaa and the recess portion 2115Ea are fitted to each other to position the transducer section 2110E to the housing 2111E. Accordingly, the ultrasound transducer unit 211E is an ultrasound transducer unit in which a transducer section is accurately positioned to a housing.

Note that, in the first embodiment described above, the endoscope in which the ultrasound transducer unit is disposed in the insertion section inserted into a subject's body has been described, but the endoscope is not limited to the above description. The configurations described above may be applied to general ultrasound probes. For example, the configurations described above may be applied to extracorporeal ultrasound probes having a function of transmitting an ultrasound pulse into a subject's body from the body surface, receiving an ultrasound echo reflected from inside the subject's body, and outputting an echo signal.

Furthermore, in the first embodiment described above, the ultrasound transducer unit having the convex ultrasound transducer in which linear piezoelectric elements are arranged into a circular shape has been described, but the ultrasound transducer unit is not limited to the above description. The above configurations may be applied to linear or radial ultrasound transducers which require positioning of the transducer section to the housing.

The present disclosure achieves an ultrasound transducer unit with a transducer section accurately positioned to a housing, an ultrasound probe, and a method of producing an ultrasound transducer unit.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound transducer unit comprising:
a transducer section including an ultrasound transducer including a plurality of piezoelectric elements arranged in an array; and
a housing configured to house the transducer section such that the transducer section is rotatable along an arrangement direction of the plurality of piezoelectric elements.

2. The ultrasound transducer unit according to claim 1, wherein
the transducer section includes a protruding portion that protrudes from at least one of side surfaces at both ends of the piezoelectric elements of the transducer section in a direction perpendicular to the arrangement direction of the plurality of piezoelectric elements, and
the housing includes a supporting portion configured to support the protruding portion.

3. The ultrasound transducer unit according to claim 1, wherein
the housing includes a protruding portion that protrudes towards at least one of side surfaces at both ends of the piezoelectric elements of the transducer section in a direction perpendicular to the arrangement direction of the plurality of piezoelectric elements, and
the transducer section includes a supporting portion configured to support the protruding portion.

4. The ultrasound transducer unit according to claim 1, wherein the supporting portion is formed on an inner wall surface of an opening portion, the opening portion is formed in the housing to house the transducer section, and the inner wall surface is perpendicular to the protruding portion and facing the protruding portion.

5. The ultrasound transducer unit according to claim 1, wherein
the protruding portion is formed on at least one of a pair of inner wall surfaces of an opening portion, the opening portion is formed in the housing to store the transducer section, and the pair of inner wall surfaces extends in the arrangement direction of the piezoelectric elements, and
the supporting portion is formed on one of side surfaces at both ends of the piezoelectric elements of the transducer section, and the one side surface faces the protruding portion.

6. The ultrasound transducer unit according to claim 1, wherein
the plurality of piezoelectric elements are arcuately arranged, and
the protruding portion and the supporting portion are arranged at a center of curvature of the arcuately arranged piezoelectric elements.

7. The ultrasound transducer unit according to claim 1, wherein
the protruding portion is formed on at least one of side surfaces at both ends of the piezoelectric elements of an acoustic lens provided on an outer surface of the ultrasound transducer, and
the supporting portion is formed on an inner wall surface of an opening portion, the opening portion is formed in the housing to house the transducer section, and the inner wall surface is perpendicular to the protruding portion and facing the protruding portion.

8. The ultrasound transducer unit according to claim 1, wherein
the protruding portion is formed on at least one of a pair of inner wall surfaces of an opening portion, the opening portion is formed in the housing to house the transducer section, and the pair of inner wall surfaces extends in the arrangement direction of the piezoelectric elements, and
the supporting portion is formed on one of side surfaces at both ends of the piezoelectric elements of an acoustic lens provided on an outer surface of the ultrasound transducer, and the one side surface faces the protruding portion.

9. An ultrasound probe comprising
the ultrasound transducer unit according to claim 1.

10. A method of producing an ultrasound transducer unit, comprising:
causing a supporting portion to rotatably support a columnar protruding portion, the columnar protruding portion being formed in one of a transducer section and a housing, the transducer section including an ultrasound transducer including a plurality of piezoelectric elements arranged in an array, the housing being configured to house the transducer section, the columnar protruding portion protruding in a direction perpendicular to an arrangement direction of the plurality of piezoelectric elements, the supporting portion being formed in another one of the transducer section and the housing to support the protruding portion;
positioning the transducer section by rotating the transducer section relative to the housing; and
fixing the positioned transducer section to the housing.

11. The method of producing an ultrasound transducer unit according to claim 10, wherein
the protruding portion is formed on at least one of side surfaces at both ends of the piezoelectric elements of the transducer section, and
the supporting portion is formed on an inner wall surface of an opening portion, the opening portion is formed in the housing to store the transducer section, and the inner wall surface is perpendicular to the protruding portion and facing the protruding portion.
